(19) United States
(12) Patent Application Publication
(10) Pub. No.: US 2003/0023254 A1
(43) Pub. Date: Jan. 30, 2003

(54) Blood vessel fastening knot in intracavitary surgery

(76) Inventor: Wen-Hsiang Chiu, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/910,782

(22) Filed: Jul. 24, 2001

(51) Int. Cl. A61B 17/08

(52) U.S. Cl. 606/158

(57) ABSTRACT

A blood vessel fastening knot used in intracavitary surgery, pre-tied in multiple loops at the central of a section of surgical wire, and temporary bound and fixed in position allowing later fastening with a size of the loops merely for a wire carrier to pass through so one end of the wire as guided by the carrier may wrap up a blood vessel and pass through said knot to release the knot by pulling outwardly both ends of the wire for the wire to fasten both of the blood vessel and the knot simultaneously.

### BACKGROUND OF THE INVENTION

1. (a) Field of the Invention

The present invention relates to a blood vessel fastening knot used in intracavitary surgery, and more particularly to one a running knot pre-tied in multiple loops and held in position before allowing surgical wire pass through by means of a certain aid to fasten the blood vessel during operation.

2. (b) Description of the Prior Art

Whereas surgery becomes more precise and accurate, the cutter gets smaller for faster cure. Operations taking advantage of endoscope in long and narrow capsule even eliminates use of suture. Furthermore, in operation involving internal organ, certain blood vessels connecting organs usually have to be cut off with the end of the blood vessel blocked. Currently, a special instrument must be used for that blocking purpose during operation. The use of such instrument comes very handy, but never an economic way since the cost is comparatively high. Use of one type of homeostatic forceps may cost as much as US $350, a heavy burdens either payable by the patient or by National Heath Insurance Program.

### SUMMARY OF THE INVENTION

The primary purpose of the present invention is to provide a blood vessel fastening knot to be used in medical surgery. To achieve the purpose, a surgical wire is pre-tied and held in position a running knob for later use to fasten a blood vessel in the operation by means of a certain aid to permit the wire to pass through the knot. Wherein, a running knot comprised of multiple loops is pre-tied at the central of a section of surgical wire, and the knot is bound and held in position before the use. The size of the loops is large enough to allow a wire carrier for operation to pass through. One end of the wire as guided by the carrier wraps up a target blood vessel and passes through the knot while a section of the wire extends for a sufficient length from the other end. By pulling outward both ends of the wire after the wire has passed through the knot, the binding status of the knot is destroyed to progressively reduce the size of the loops and thus to fasten both of the knot and the blood vessel at the same time.

### BRIEF DESCRIPTION OF THE DRAWINGS

FIG. 1 is a schematic view showing a structure of a preferred embodiment of the present invention.

FIG. 2 is a first view showing the preferred embodiment in operation.

FIG. 3 is a second view showing the preferred embodiment in operation.

FIG. 4 is a third view showing the preferred embodiment in operation.

FIG. 5 is a fourth view showing the preferred embodiment in operation.

FIG. 6 is a fifth view showing the preferred embodiment in operation.

FIG. 7 is a schematic view showing a use of the preferred embodiment in its completed status.

### DETAILED DESCRIPTION OF THE PREFERRED EMBODIMENTS

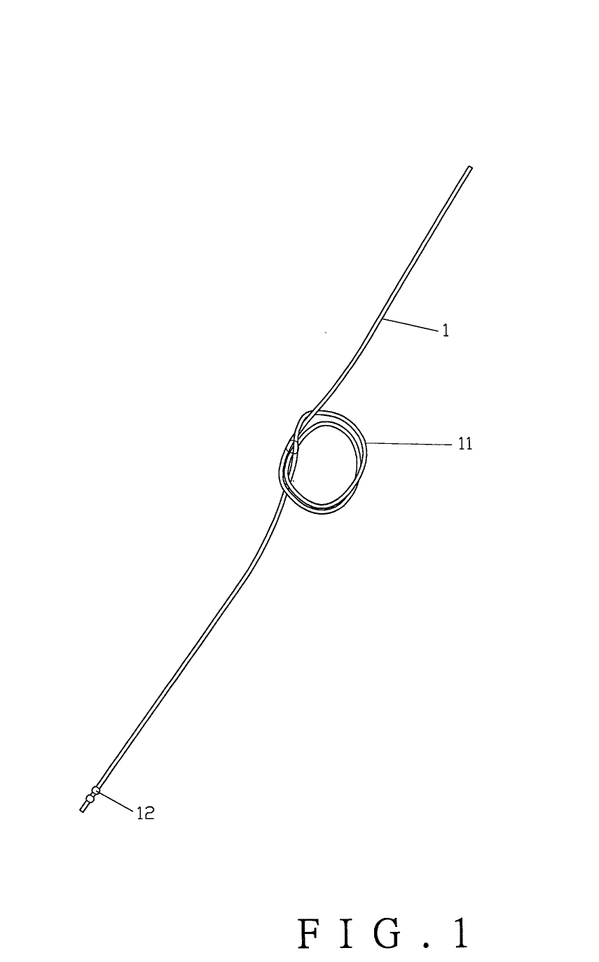

Referring to FIG. 1 for a preferred embodiment of the present invention, wherein, a blood vessel fastening knot relates to a section of a surgical wire (1) pre-tied a running knot and held in position before the use in a configuration characterized by that:

a running knot (11) is pre-tied at the central section of the wire (1) in multiple loops as illustrated. The knot (11) is temporarily bound in position so that such binding status can be destroyed with a minimum externally applied force to become a running knot for progressively diminishing its loop size. Furthermore, size of said knot (11) merely permits a wire carrier (2) to pass through, and a retaining member (12) is provided at one end of the wire (1) where to guide the wire (1) to pass through the knot (11) by a wire carrier (2) and to relatively hold the active end of the wire carrier (2) while a section of the wire (1) extends in a proper length from the knot (11) towards the other end of the wire (1).

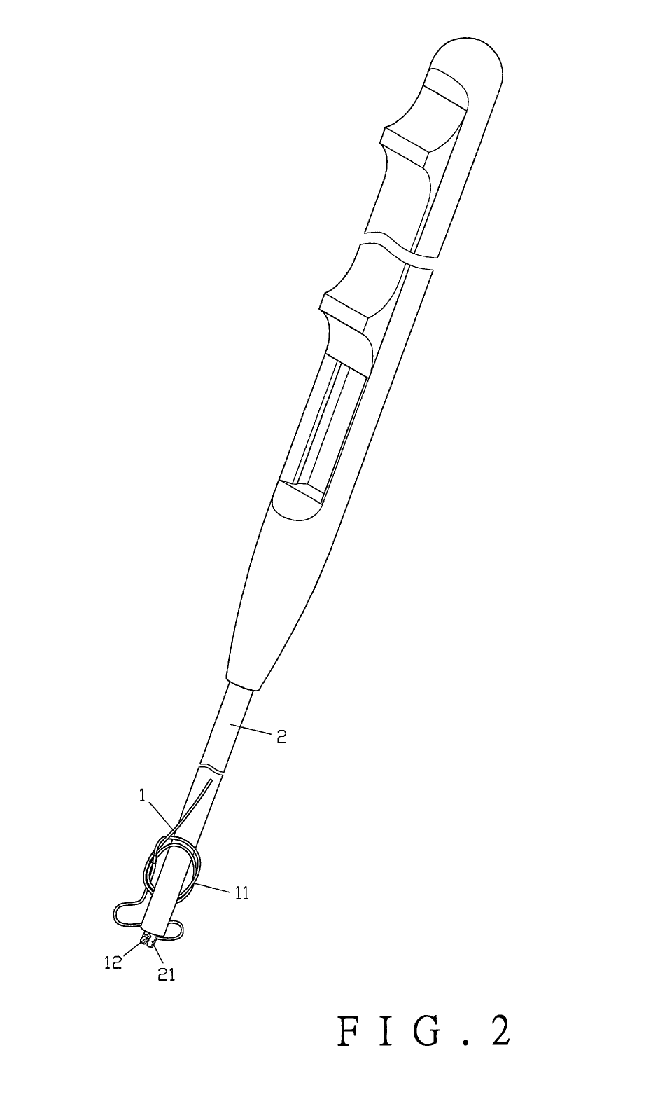

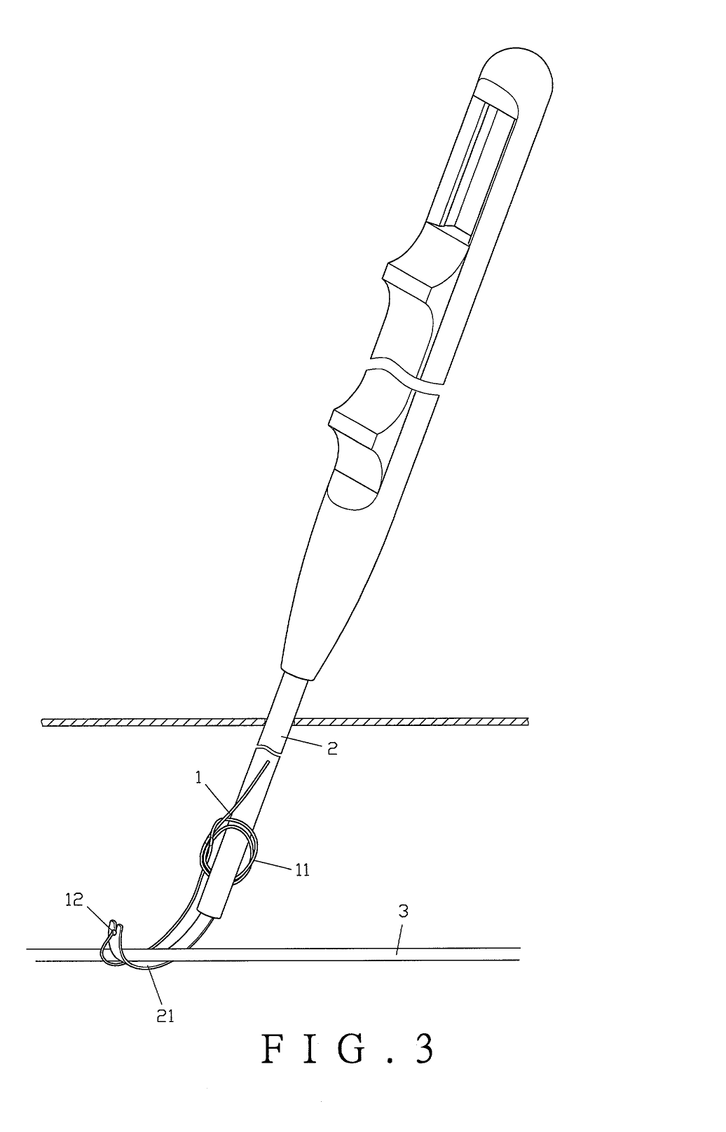

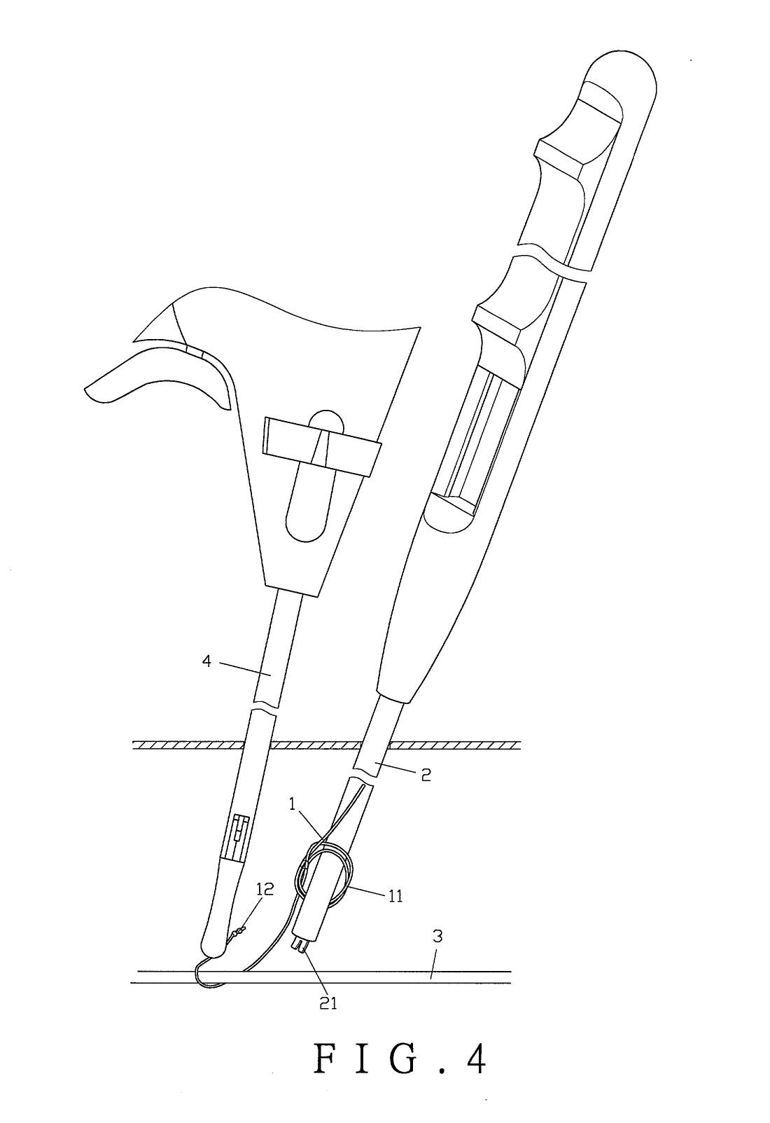

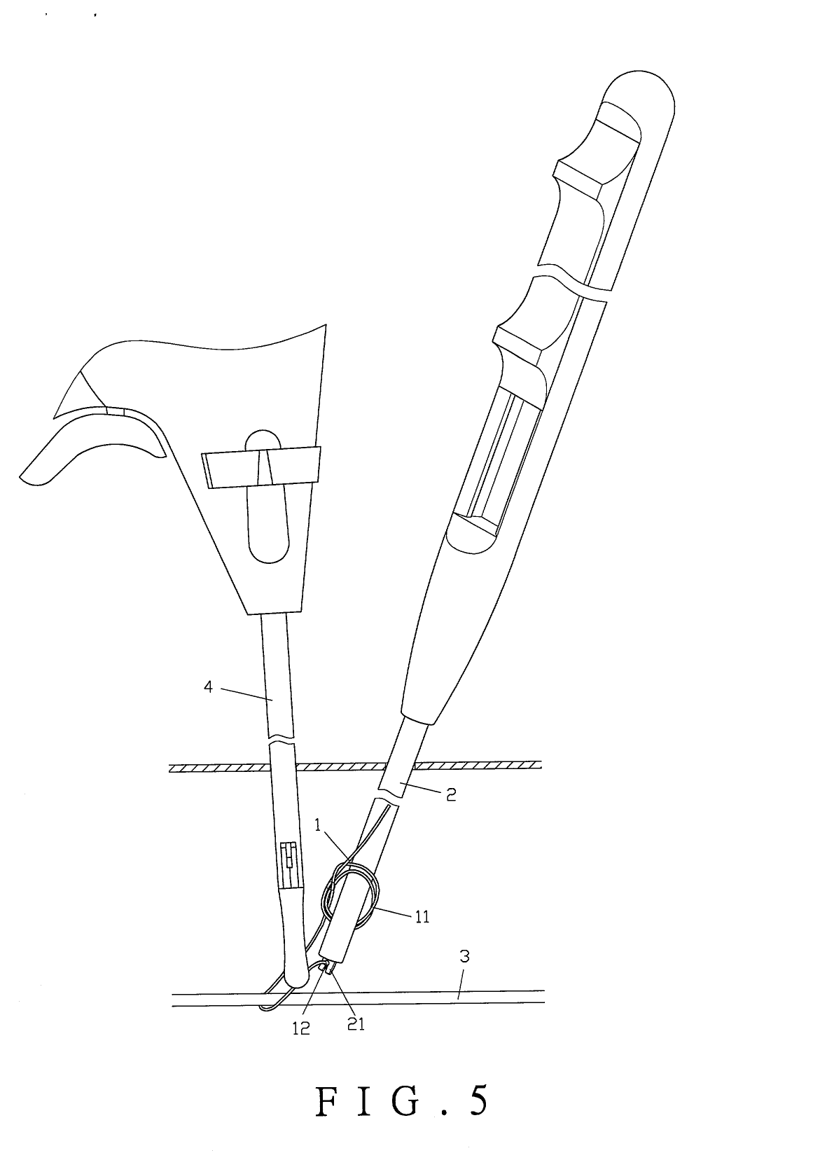

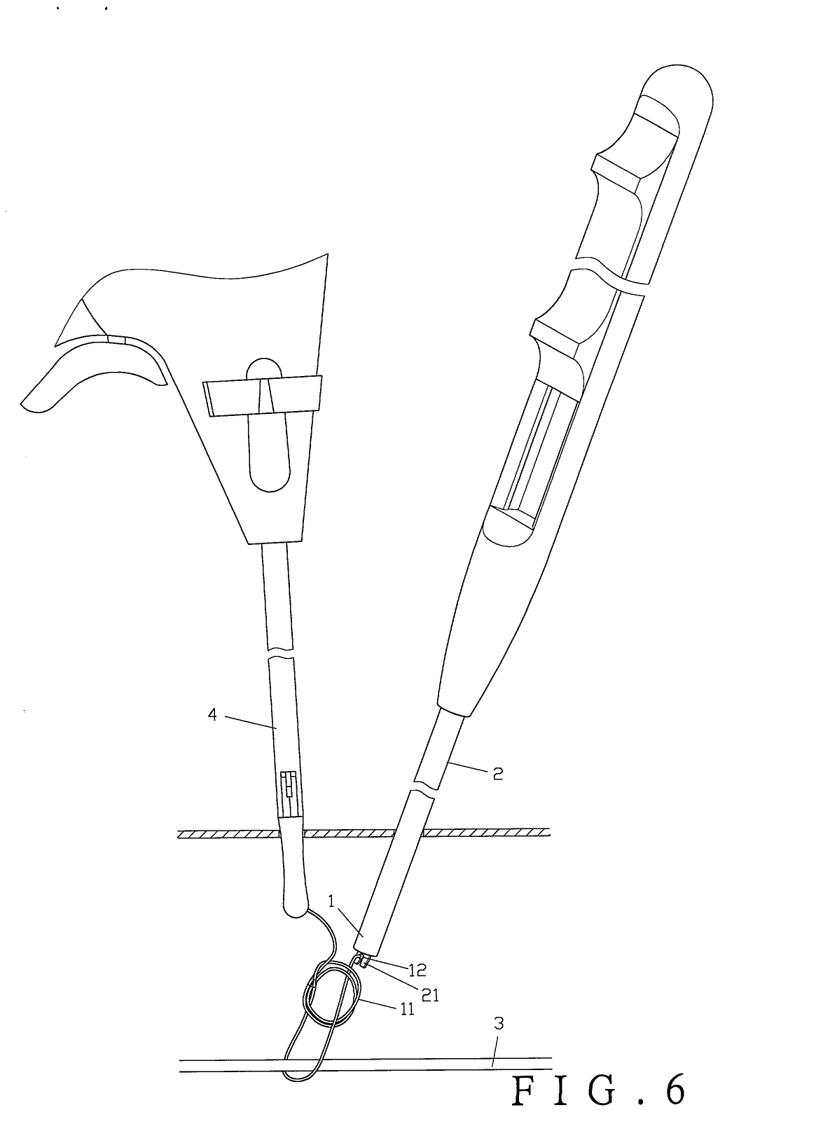

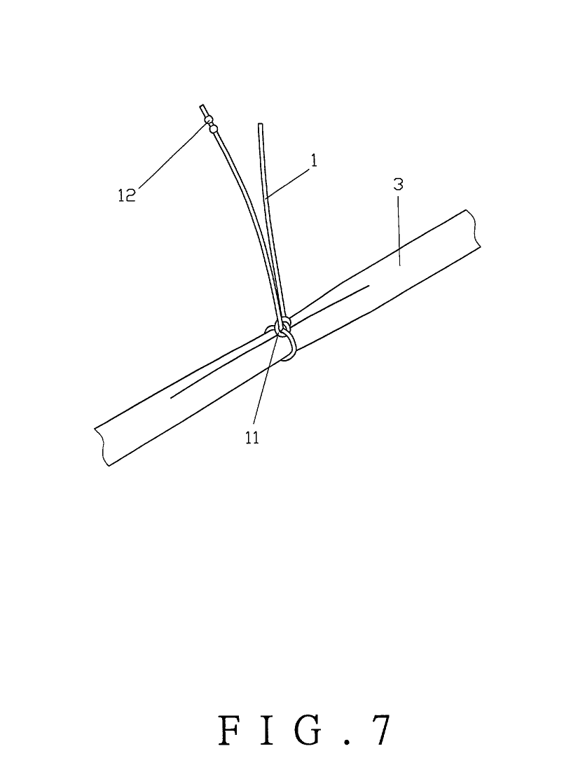

The wire (1) with the pre-tied knot (11) held in position before the use becomes a product that can be used to fasten a blood vessel during a surgical operation. In the operation, the wire carrier (2) passes through the knot (11) of the wire (1) before cutting off a target blood vessel. As illustrated in FIG. 2, a retractable inner clip (21) protruding from the front of the wire carrier (2) clips the retaining member (12) at one end of the wire (1). Now referring to FIG. 3, both of the wire (1) and the wire carrier (2) pick up a blood vessel so that the end of the wire (1) provided with the retaining member wraps up the blood vessel (3) as guided by the inner clip (21) when retracted. As illustrated in FIG. 4, a clamp (4) is inserted to separate the retaining member (12) of the wire (1) from the end of the inner clip (21) of the wire carrier (2). The inner clip (21) immediately retracts into the wire carrier (2) and the wire carrier (2) is slightly pulled back. As illustrated in FIG. 5, the wire carrier (2) once again picks up with its inner clip (21) the end of the retaining member (12) of the wire (1) in conjunction with the use of the clamp (4). Then, as illustrated in FIG. 6, the wire carrier (2) retreats from the knot (11) to guide one end of the wire (1) to wrap up the blood vessel and pass through the knot (11). Finally, with the clamp (4) firmly holding another end of the wire (1), both ends of the wire (1) are pulled outwardly to destroy the binding status of the knot (11). The running knot (11) progressively reduces in size down to such as illustrated in FIG. 7 to fasten both of the blood vessel (3) and the knot (11) without getting loose at their own. Depending on the actual needs, more than one knots of the present invention may be correspondingly used before cutting blood vessels in between.

The wire and the clamp disclosed in the preferred embodiment of the present invention are not restrictive since other types of surgical instruments may be used in conjunction with the knot of the present invention as long as they permit wire passing and pulling as required by the operation.

A blood vessel fastening knot for the intracavitary operation as disclosed above of the present invention features a pre-tied running knot held in position before the use to provide a blood vessel fastening pattern by means of a certain aid of wire carrier. The wire with the pre-tied knot cm be easily produced at cheaper cost, and is convenient in use. No identical product to the present invention has been used in the intracavitary surgery. In addition to being innovative, a significant cost reduction to upgrade economic benefits can be realized by the present invention. Therefore, an application for utility patent is duty filed accordingly.

I claim:

1. A blood vessel fastening knot to be used in intracavitary surgery essentially comprised of a pre-tied running knot provided at the center of a section of wire to allow a surgical wire carrier to pass through said knot so that one end of the wire being guided by said wire carrier to wrap up a blood vessel and pass through said knot while a section of the wire at the other end extends for a proper length; then both ends are pulled outwardly for the wire to fasten both of the knot and the blood vessel at the same time.

2. A blood vessel fastening knot to be used in intracavitary surgery as claimed in claim 1, wherein, the knot to allow the wire carrier pass through may be made in two or more than two loops.

3. A blood vessel fastening knot to be used in intracavitary surgery as claimed in claim 1, wherein, the knot being bound in position before the use with said binding status could be destroyed with a minimum externally applied force.

4. A blood vessel fastening knot to be used in intracavitary surgery as claimed in claim 1, wherein, a retaining member is provided at the end of the wire for the wire carrier to guide the wire to pass through the knot to fasten the blood vessel.
